(19) United States
(12) Patent Application Publication
(10) Pub. No.: US 2021/0186977 A1
(43) Pub. Date: Jun. 24, 2021

(54) MEDICAL USES

(71) Applicant: XNK THERAPEUTICS AB, Huddinge (SE)

(72) Inventors: Hareth Nahi, Alvsjo (SE); Carl Arnold Gosta Gahrton, Bromma (SE); Evren Alici, Huddinge (SE); Hans-Gustaf Einar Ljunggren, Stockholm (SE)

(21) Appl. No.: 17/048,702

(22) PCT Filed: Apr. 30, 2019
(86) PCT No.: PCT/EP2019/061122

§ 371 Date: Oct. 19, 2020

(30) Foreign Application Priority Data
Apr. 30, 2018 (GB) 1807050.8

(51) Int. Cl. A61K 31/522; A61K 31/52; A61P 35/00; A61K 35/17; A61K 9/00; C12N 5/0783

(52) U.S. Cl. CPC A61K 31/522; A61K 31/52; A61P 35/00; C12N 5/0636; A61K 9/0053; A61K 9/0019; A61K 35/17

(57) ABSTRACT

The present invention relates generally to anti-viral agents for use in preventing herpes virus reactivation in a patient, wherein the patient has received a therapy comprising natural killer (NK) cells and/or NK-like T cells. Additionally, the invention relates to NK cells and/or NK-like T cells for use in treating a malignant disease in a patient, wherein the use comprises the step of administering an anti-viral agent to the patient with the NK cell and/or NK-like T cell therapy. The invention also relates to pharmaceutical compositions and kits.

The present invention relates generally to anti-viral agents for use in preventing herpes virus reactivation in a patient, wherein the patient has received a therapy comprising natural killer (NK) cells and/or NK-like T cells. Additionally, the invention relates to NK cells and/or NK-like T cells for use in treating a malignant disease in a patient, wherein the use comprises the step of administering an anti-viral agent to the patient with the NK cell and/or NK-like T cell therapy. The invention also relates to pharmaceutical compositions and kits.

Multiple myeloma (MM) is a malignant neoplasm characterized by clonal proliferation of plasma cells in the bone marrow. MM is still considered incurable due to the persistence of minimal residual disease (MRD), which is potentially due to the MM cells remaining after treatment (Alici E, Bjorkstrand B, Treschow A, Aints A, Smith C I, Gahrton G, Dilber M S. Long-term follow-up of gene-marked CD34+ cells after autologous stem cell transplantation for multiple myeloma. Cancer Gene Ther. 2007; 14(3):227-32).

The use of cellular immunotherapy against cancer has been investigated since the introduction of lymphokine-activated killer (LAK) cells in the mid-1980s (Grimm E A. et al., 1982; Rosenberg S., 1985). Adoptive transfer of cytotoxic effector cells with tumor cell-killing potential in order to induce a graft-versus-tumor effect has been an attractive approach against cancer. Natural killer (NK) and NK-like T cells constitute a relatively high cytotoxic capacity among other effector-cell populations having a potential antitumor effect (3). However, the low percentages of these cells in peripheral blood mononuclear cells (PBMCs) and effector-cell preparations, such as LAK cells, represent a barrier for their use in clinical trials and use as a cancer therapy p The inventors' previous studies demonstrated that long-term ex vivo expansion and activation of autologous NK cells from MM patients can provide significantly superior cytotoxic activity against autologous tumor cells when compared to short-term activated autologous NK cells (Alici E, Sutlu T, Bjorkstrand B, Gilljam M, Stellan B, Nahi H, et al. Autologous antitumor activity by NK cells expanded from myeloma patients using GMP-compliant components. Blood. 2008; 111(6):3155-62 and Sutlu T, Stellan B, Gilljam M, Quezada H C, Nahi H, Gahrton G, et al. Clinical-grade, large-scale, feeder-free expansion of highly active human natural killer cells for adoptive immunotherapy using an automated bioreactor. Cytotherapy. 2010; 12(8):1044-55). The inventors have also reported efficient NK cell-based treatment of MM development in an animal model (Alici E, Konstantinidis K V, Sutlu T, Aints A, Gahrton G, Ljunggren H G, et al. Anti-myeloma activity of endogenous and adoptively transferred activated natural killer cells in experimental multiple myeloma model. Exp Hematol. 2007; 35(12):1839-46.). Having developed a procedure for NK cell expansion in a closed-automated bioreactor using clinical grade good manufacturing practices (GMP)-compliant components, the inventors were given approval from the Swedish Medicinal Products Agency (EudraCT: 2010-022330-83) and the ethical committees (EPN: 2013/490-32) to initiate a first-in-man phase I/II clinical trial (Sutlu T, Stellan B, Gilljam M, Quezada H C, Nahi H, Gahrton G, et al. Clinical-grade, large-scale, feeder-free expansion of highly active human natural killer cells for adoptive immunotherapy using an automated bioreactor. Cytotherapy. 2010; 12(8):1044-55 and Sutlu T, Alici E. Ex vivo expansion of natural killer cells: a question of function. Cytotherapy. 2011).

Very surprisingly, during the clinical trial the inventors observed a reactivation of a herpes virus (specifically varicella zoster virus (VZV), which manifested as shingles), in patients who received NK cell and/or and NK-like T cell therapy. As is well-known, shingles and other conditions caused by herpes virus reactivation and/or infection are unpleasant conditions, and in the present context greatly reduce the quality of life of patients that are already seriously unwell and receiving NK cell and/or NK-like T cell therapy.

The inventors' surprising findings suggest new approaches for using and managing NK cell and/or NK-like T cell therapy.

Accordingly, in a first aspect, the invention provides an anti-viral agent for use in preventing a herpes virus reactivation in a patient, wherein the patient has received a therapy comprising natural killer (NK) cells and/or NK-like T cells, wherein the use comprises the step of administering the anti-viral agent to the patient with the NK cell and/or NK-like T cell therapy.

In a second aspect, the invention provides use of an anti-viral agent in the manufacture of a medicament for preventing a herpes virus reactivation in a patient, wherein the patient has received a therapy comprising natural killer (NK) cells and/or NK-like T cells, wherein the anti-viral agent is administered to the patient with the NK cell and/or NK-like T cell therapy.

In a third aspect, the invention provides a method for preventing a herpes virus reactivation in a patient, wherein the patient has received a therapy comprising natural killer (NK) cells and/or NK-like T cells, wherein the method comprises the step of administering the anti-viral agent to the patient with the NK cell and/or NK-like T cell therapy.

By “herpes virus” we include the large family of DNA viruses known as Herpesviridae (or herpesviruses). There are nine herpesvirus types known to infect humans: herpes simplex viruses 1 and 2 (HSV-1 and HSV-2), (also known as human herpesvirus 1 (HHV-1) and HHV2), varicella-zoster virus (VZV or HHV-3), Epstein-Barr virus (EBV or HHV-4), human cytomegalovirus (HCMV or HHV-5), human herpesvirus 6A and 6B (HHV-6A and HHV-6B), human herpesvirus 7 (HHV-7), and Kaposi's sarcoma-associated herpesvirus (KSHV, also known as HHV-8). In total, there are more than 130 herpes viruses (Whitley RJ. Herpesviruses. In: Baron S, editor. Medical Microbiology. 4th edition. Galveston (TX): University of Texas Medical Branch at Galveston; 1996. Chapter 68.).

At least five species of herpes viruses—namely HSV-1 and HSV-2 (both of which can cause orolabial herpes and genital herpes), varicella zoster virus (the cause of chickenpox and shingles), Epstein-Barr virus (implicated in several diseases, including mononucleosis and some cancers), and cytomegalovirus—are extremely widespread among humans. More than 90% of adults have been infected with at least one of these, and a latent form of the virus remains in most people.

The term “Varicella-Zoster virus (VZV)” is used to describe a virus which causes varicella (chickenpox) and herpes zoster (shingles). Varicella results from a primary infection with the virus; herpes zoster results from secondary invasion by the same virus or by reactivation of infection which in many instances may have been latent for a number of years.

As discussed above and in the accompanying Examples, the inventors surprisingly identified the reactivation of VZV in patients receiving a therapy comprising natural killer (NK) cells and/or NK-like T cells. VZV is a double-stranded DNA virus and it is morphologically identical with herpes simplex viruses. It is a causative agent for both chickenpox and herpes zoster (shingles) which is characterized by an inflammatory reaction of the posterior nerve roots and ganglia, accompanied by the affected sensory nerves. Chickenpox follows initial exposure to the virus and is typically a relatively mild, self-limited childhood illness with a characteristic exanthem, but can become disseminated in immunocompromised children. Even when clinical symptoms of chickenpox have resolved, VZV remains dormant in the nervous system of the infected person (also called virus latency), in the trigeminal and dorsal root ganglia.

VZV reactivation later in life produces a disease known as herpes zoster or shingles. Serious complications of shingles include post-herpetic neuralgia (PHN), zoster multiplex, myelitis, herpes ophthalmicus, or zoster sine herpete. A common complication of shingles is post-herpetic neuralgia (PHN), a chronic, often debilitating pain condition that can last months or even years. The risk for PHN in patients with shingles is 10%-18%.

Pain and paraesthesia are typically the first symptoms of VZV infection. Until the characteristic vesicular rash erupts, diagnosis may be difficult. A prodromal period during which symptoms may vary is common. Pain, itching and paraesthesia are common symptoms.

During the acute illness, patients may experience the following: pain, helplessness and depression and/or flulike symptoms. The most common presentation is the shingles vesicular rash, which most commonly affects a thoracic dermatome, after a prodromal illness of pain and paraesthesia, erythematous macules and papules develop and progress to vesicles within 24 hours. The vesicles eventually crust and resolve. Pain and sensory loss are the usual symptoms, motor weakness also occurs and is frequently missed on examination. In severe cases actual monoplegia due to VZV brachial plexus neuritis have been reported (Prevention of herpes zoster: recommendations of the Advisory Committee on Immunization Practices (ACIP). MMWR Recomm Rep. 2008; 57(05):1-30).

The term “Herpes Simplex Virus” (HSV) is used to describe HSV1 and HSV 2 which are the causative viral agents of herpes simplex infections.

HSV1 and 2 have about 50 percent genomic homology but share most other characteristics. Manifestations of herpes simplex virus infection include: gingivostomatitis, herpes genitalis, herpetic keratitis, and dermal whitlows. Neonatal herpes simplex virus infection and herpes simplex virus encephalitis also occur.

The virus replicates initially in epithelial cells, producing a characteristic vesicle on an erythematous base. It then ascends sensory nerves to the dorsal root ganglia, where, after an initial period of replication, it establishes latency. During reactivated infection, the virus spreads distally from the ganglion to initiate new cutaneous and/or mucosal lesions. HSV1 transmission is primarily oral, and herpes simplex virus 2 primarily genital. Transmission requires intimate contact (Whitley R J. Herpesviruses. In: Baron S, editor. Medical Microbiology. 4th edition. Galveston (Tex.): University of Texas Medical Branch at Galveston; 1996. Chapter 68.).

The term “Epstein-Barr virus (EBV)” is used to describe a herpesvirus found in cell cultures of Burkitts lymphoma. EBV is the causative agent in infectious mononucleosis, as well as in a number of other related conditions/disease states, including EBV-associated lymphomas. Epstein-Barr virus causes classic mononucleosis. In immunocompromised hosts, the virus causes a lymphoproliferative syndrome. In some families, Epstein Barr virus causes Duncan's syndrome.

Epstein Barr virus replicates in the epithelial cells of the oropharynx and in β lymphocytes. Epstein Barr virus is transmitted by intimate contact, particularly via the exchange of saliva (Whitley R J. Herpesviruses. In: Baron S, editor. Medical Microbiology. 4th edition. Galveston (Tex.): University of Texas Medical Branch at Galveston; 1996. Chapter 68.).

The term “Cytomegalovirus (CMV)” is used to describe an infection which is typically unnoticed in healthy people, but can be life-threatening for the immunocompromised, such as HIV-infected persons, organ transplant recipients, or new born infants. It is found in a significant proportion of the population. As with EBV, seropositivity increases with age. Ganciclovir, which inhibits the replication of all human herpes viruses, is usually used to treat CMV, especially to treat retinitis. Foscarnet is also approved in the US. Cytomegalovirus causes three clinical syndromes: (1) Congenital cytomegalovirus infection (when symptomatic) causes hepatosplenomegaly, retinitis, rash, and central nervous system involvement; (2) In about 10 per cent of older children and adults, primary cytomegalovirus infection causes a mononucleosis syndrome with fever, malaise, atypical lymphocytosis, and pharyngitis; (3) Immunocompromised hosts (transplant recipients and human immunodeficiency virus [HIV]-infected individuals) may develop life-threatening disseminated disease involving the lungs, gastrointestinal tract, liver, retina, and central nervous system.

Cytomegalovirus replicates mainly in the salivary glands and kidneys and is shed in saliva and urine. Replication is slow, and the virus induces characteristic giant cells with intranuclear inclusions. Transmission is via intimate contact with infected secretions. Cytomegalovirus infections are among the most prevalent viral infections worldwide (Whitley R J. Herpesviruses. In: Baron S, editor. Medical Microbiology. 4th edition. Galveston (Tex.): University of Texas Medical Branch at Galveston; 1996. Chapter 68.).

As is well known, infection with a virus is initiated when a viral particle contacts a cell with specific types of receptor molecules on the cell surface. Following binding of viral envelope glycoproteins to cell membrane receptors, the virion is internalized and dismantled, allowing viral DNA to migrate to the cell nucleus. Within the nucleus, replication of viral DNA and transcription of viral genes occurs. During symptomatic infection, infected cells transcribe lytic viral genes.

The herpes viruses are known to exist latently in hosts, where they can reside for many years without any apparent sign of infection. In such cells, a small number of viral genes termed latency associated transcript (LAT) accumulate. During this latent lysogenic cycle, the virus can remain asymptomatically dormant (or latent) in the ganglia adjacent to the spinal cord (called the dorsal root ganglion) and/or the trigeminal ganglion in the base of the skull. By remaining dormant (latent and/or inactive), the virus can persist in the cell (and thus the host) indefinitely. While primary infection is often accompanied by a self-limited period of clinical illness, long-term latency is symptom-free.

By “herpes virus reactivation” we include the meaning of a non-primary infection such as the reactivation of a latent and/or dormant and/or inactive and/or endogenous herpes virus in a patient which can lead to a herpes virus infection. This can include the onset of conditions or diseases associated with latent herpes virus infections in a patient (such as shingles). We include a herpes virus selected from the group comprising: varicella zoster virus (VZV); herpes simplex virus (HSV) type 1 and 2; Epstein Barr Virus (EBV); and cytomegalovirus (CMV). In a preferred embodiment, the herpes virus is VZV.

Reactivation of latent viruses has been implicated in a number of diseases (e.g. shingles and Pityriasis Rosea). Following reactivation, transcription of viral genes transitions from latency-associated LAT to multiple lytic genes; these lead to enhanced replication and virus production. Often, lytic activation leads to cell death. Clinically, lytic activation is often accompanied by emergence of non-specific symptoms such as low grade fever, headache, sore throat, malaise, and rash as well as clinical signs such as swollen or tender lymph nodes.

By “a patient” we include the meaning of a subject receiving or intended to receive medical treatment and/or prophylaxis, or a subject in need of treatment and/or prevention of herpes virus reactivation. The patient may be a vertebrate, such as a vertebrate mammal, for example a human, or a non-human mammal, such as a domestic animal (for example, cat, dog, rabbit, cow, sheep, pig, mouse or other rodent). Preferably the patient is human.

By “anti-viral agent” we include any synthetic or natural molecule or compound that is capable of preventing reactivation of a herpes virus in the patient. Such agents may exert an antiviral effect by, for example, inactivating extracellular virus particles and/or preventing viral attachment and/or cellular entry and/or, preventing replication of the viral genome and/or preventing synthesis of specific viral protein(s) and/or preventing assembly and/or release of new infectious virions. Examples of known anti-viral agents include nucleoside analogues after phosphorylation to their triphosphate forms and phosphonoformic and phosphonoacetic acids and their analogues.

It will be appreciated that the anti-viral agent is for use in the prevention of herpes virus reactivation in a patient.

In an embodiment the anti-viral agent is a vaccine.

In an alternative embodiment the anti-viral agent is not a vaccine.

By “preventing a herpes virus reactivation” we include fully or partially preventing, suppressing and/or reducing the reactivation of a herpes virus infection and/or conditions or diseases associated with latent herpes virus infections in a patient (such as shingles). Prevention of reactivation may include the prevention of reactivation of herpes lying dormant in neural tissue and/or the prevention of occurrence of symptoms in an infected patient and/or a decrease in severity or frequency of symptoms of viral reactivation, or a condition or disease caused by virus reactivation in the patient.

In an embodiment the patient is susceptible to herpes virus reactivation. In a further embodiment, the patient is susceptible to the development of shingles.

If herpes virus reactivation is completely prevented, the patient will be asymptomatic for viral infection. In some embodiments the anti-viral agent may eradicate part of the latent viral reservoir leading to a reduction in the proportion of reactivable virus and therefore preventing, suppressing and/or reducing herpes virus reactivation. If herpes virus reactivation is reduced and/or suppressed it may shorten the duration of clinical manifestations (such as, for example, headache, burning, tingling, numbness or itchiness of the skin in the affected area, a feeling of being generally unwell, a high temperature (fever) and a rash that can develop into itchy blisters).

By “NK cell and/or NK-like T cell therapy” we include the administration of NK cells and/or NK-like T cells to a patient for therapeutic purposes. The patient may have a malignant disease such as a haematological cancer, a solid tumour, or a chronic viral infection, or be another patient in need of such therapy.

It will be appreciated that the term “NK cell and/or NK-like T cell therapy” refers to a therapy comprising a therapeutically effective amount of NK cells and/or NK-like T cells.

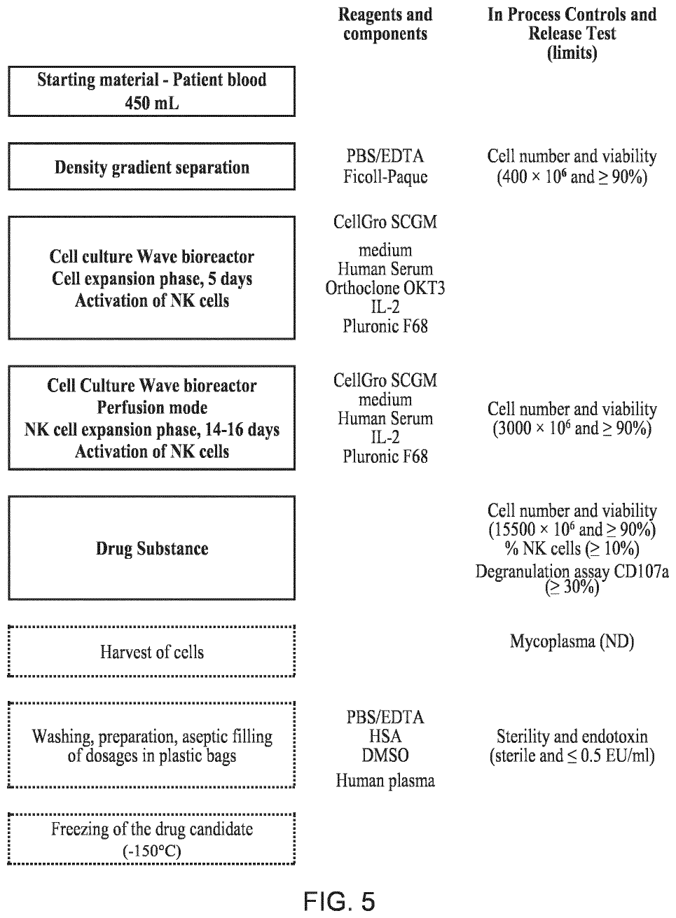

It will be appreciated that NK cell and/or NK-like T cell therapy is a form of adoptive cell transfer (ACT), i.e. the transfer of cells into a patient, and the two terms may be used interchangeably herein. In a preferred embodiment, the cells originate from the patient (autologous). In an alternative embodiment, the cells originate from another individual (heterologous). The terms “NK cell and/or NK-like T cell therapy” and “CellProtect” may be used interchangeably herein. The protocol for making CellProtect is described below and depicted in FIG. 5.

In a preferred embodiment, the NK cell and/or NK-like T cells have been expanded and activated ex vivo and are administered to a patient in need thereof. The preparation of NK cell and/or NK-like T cells is described in earlier publication WO 2010/110734, incorporated herein by reference.

In an embodiment, the NK cell and/or NK-like T cell therapy comprises at least 10% NK cells with the phenotype CD3-CD56+. In an embodiment, at least 30% of the NK cells are activated NK cells.

The NK cells and/or NK like T cells can be administered by infusion, for example through a central-vein catheter, or intravenously (IV), or into the cerebrospinal fluid in order for it to reach the central nervous system (CNS). Administration can be intratumoral (i.e. injection directly into the tumour), for example, into the tumour cavity.

In a preferred embodiment the patient is not lymphodepleted. By “the patient is not lymphodepleted” we include the meaning of a patient who has not received lymphodepletion. Lymphodepletion is a non-selective method of depleting (i.e. eliminating) lymphocytes, such as T cells, for example, regulatory T cells.

Lymphodepletion can be accomplished by any means known in the art, including total body irradiation, chemotherapy, or as a result of a disease process, such as leukemia or HIV/AIDS. Alternatively, lymphodepletion can be accomplished by administering an antibody which specifically binds to lymphocytes. Lymphopenia and lymphodepletion are used interchangeably to describe the state of reduced lymphocyte number. In particular embodiments, a patient is lymphodepleted if the number of lymphocytes in the patient decreases by at least 50%, such as at least 60%, 70%, 80% or 90%, following administration of a lymphodepletion agent.

By “administering the anti-viral agent to the patient with the NK cell and/or NK-like T cell therapy” we include administering the anti-viral agent before, concurrently or after the patient receives NK cell and/or NK-like T cell therapy. In one embodiment the anti-viral agent is administered to the patient concurrently with NK cell and/or NK-like T cell therapy.

In a preferred embodiment the NK cell and/or NK-like T cell therapy induces and/or increases herpes virus infection.

By “the NK cell and/or NK-like T cell therapy induces and/or increases herpes virus reactivation” we include the meaning that the NK cell and/or NK-like T cell therapy is fully or partially responsible for the reactivation of a herpes virus in a patient, or fully or partially responsible for increasing the reactivation of a herpes virus in a patient. For example, the NK cell and/or NK-like T cell therapy may induce and/or increase herpes virus reactivation by at least 5-fold, or at least 10-fold, or at least 50-fold more than in a patient who has not received NK cell and/or NK-like T cell therapy.

Methods for measuring virus reactivation are well known in the art and include measuring viral copy number. In addition, quantification of antibodies to herpes viruses is commonly used as an indirect measure of herpes virus reactivation. In addition, clinical symptoms can be used to indicate herpes virus reactivation.

As described in the accompanying Examples, during a clinical trial, the inventors observed a reactivation of VZV and manifestation of shingles in a number of patients who received NK cell and/or NK-like T cell therapy. Without wishing to be bound by theory, the inventors believe that activated NK cells and/or NK-like T cells, upon adoptive cell transfer, may attack reservoir cells for herpes viruses which, in turn, due to induced stress, might cause a viral reactivation.

Accordingly, in one embodiment, the herpes virus lies dormant (latent) in the dorsal root ganglia.

In one embodiment of the invention, the patient has a malignant disease. By “malignant disease” we include a disease, including but not limited to cancer, in which the progress is rapid and generally threatening or resulting in death within a short time. We include the meaning of malignancies such as solid tumours, viral cancers and cancers selected from the group comprising or consisting of: colorectal cancer; brain cancer (such as medulloblastoma and glioblastoma); neuroblastoma; bone cancer; epithelial cell-derived neoplasia (epithelial carcinoma); basal cell carcinoma; adenocarcinoma; gastrointestinal cancer; lip cancer, mouth cancer, oesophageal cancer, small bowel cancer; stomach cancer; colon cancer; liver cancer; bladder cancer; pancreatic cancer; ovarian cancer; cervical cancer; lung cancer; breast cancer; skin cancer (such as melanoma), squamous cell and basal cell cancers; prostate cancer, renal cell carcinoma and sarcoma (such as soft tissue sarcoma).

In one embodiment of the invention, the NK cell and/or NK-like T cell therapy is for use in the treatment of a malignant disease.

In a preferred embodiment, the malignant disease is a haematological cancer. By “haematological cancer” we include types of cancer affecting blood, bone marrow and lymph nodes, such as those selected from the group comprising or consisting of: myeloma, lymphoma, leukaemia and chronic myeloproliferative diseases.

In a preferred embodiment of the invention, the haematological cancer is one selected from the group consisting of: myeloma, lymphoma, leukaemia and/or chronic myeloproliferative diseases.

In a preferred embodiment, the haematological cancer is multiple myeloma (MM).

In an embodiment, the NK cells have the phenotype CD3⁻CD56⁺ and/or NK-like T cells have the phenotype CD3⁺CD56⁺.

Preferably, the NK cell and NK-like T cells have been expanded ex vivo. For example, expansion could have taken place in a closed expansion system, such as in in cell culture bags within an automated bioreactor system (see Example 1 and FIG. 5). Less preferably the NK cell and NK-like T cells have been expanded in tissue culture flasks.

Such methods have been described previously by the inventors in WO 2010/110734 (see “1: Ex vivo expansion of NK cells and NK-like T cells from peripheral blood”) and in Alici E, et al., Blood. 2008;111(6):3155-62.

The expansion is preferably performed until the total number of cells has expanded at least about 10-fold or until at least about 50% of the expanded cell population comprises activated NK cells and NK-like T cells, respectively. For example, at least about 50% of the expanded cell population comprises NK cells with the phenotype CD3⁻CD56⁺.

In one embodiment, the NK and NK-like T cells have been activated ex vivo and become cytotoxic. In one embodiment, the NK cell and NK-like T cells have been expanded and activated simultaneously ex vivo. By “activated” we include the meaning that the NK cells and/or NK-like T cells have received an activating signal. Activated NK cells are capable of killing certain target cells with deficiencies in MHC class I expression. NK cells must receive an activating signal which can come in a variety of forms, the most important of which are cytokines, Fc-receptors or other activating receptors. Cells can also be activated to produce cytokines and chemokines.

Activated NK cells and NK-like T cells exhibit an increased cytotoxicity as determined by in vitro cytotoxicity tests. A skilled person can determine the cytotoxicity using methods known in the art.

One way of determining if cells exhibit an increased cytotoxicity is to use the in vitro analysis of cell mediated cytotoxicity against K562 cells using the standard 4 hour ⁵¹Cr-release assay. Briefly, Chromium-51 is incubated with human cancer cell for 1 h at 37 C, thrice washed and co-cultured with NK cells at three E:T ratios (20:1, 6.66:1, 2.22:1). 5% Triton-X 100 can be used to achieve total cell lysis. After 4 h, supernatant is harvested and analyzed in an Automatic Gamma Counter. Specific ⁵¹Cr lysis is calculated using the following equation: Percentage of specific lysis=100×(Test release−Spontaneous release)/(Maximal release−Spontaneous release).

Alternatively, a degranulation assay can be used. For example, a degranulation assay against K562 cells, followed by measuring the percentage of degranulated cells in each lymphocyte subpopulation. Both of these assays are described in WO 2010/110734 (see “3. Evaluation of cell mediated cytotoxicity”). Other in vitro cytotoxicity tests are known in the art.

In one embodiment, the anti-viral agent is administered before and/or concurrently and/or after the patient has received the NK cell and/or NK-like T cell therapy. It will be appreciated that it is desirable to commence administration of the anti-viral agent before the reactivation of a dormant herpes infection is sensed or suspected, that is the prodromal stage. Accordingly, preferably, the anti-viral agent is administered before and/or concurrently with NK cell infusion.

By “administered before” we include the meaning that the anti-viral agent is first administered before the patient receives NK cell and/or NK-like T cell therapy. By “administered concurrently” we include the meaning that the anti-viral agent is first administered to the patient simultaneously with the NK cell and/or NK-like T cell therapy. In another embodiment, the anti-viral agent is administered after NK cell and/or NK-like T cell therapy. By “administered after” we include the meaning that the anti-viral agent is first administered after the patient has received NK cell and/or NK-like T cell therapy.

Preferably, the anti-viral agent is administered before the appearance of the first symptoms of viral infection in the patient.

In one embodiment of the invention, the patient is administered the anti-viral agent at least one day before the patient receives the NK cell and/or NK-like T cell therapy, such as: at least two days before; or at least three days before; or at least four days before; or at least five days before; or at least six days before; or at least seven days before; or at least eight days before; or at least nine days before; or at least ten days before; or at least 20 days before; or at least 30 days before; or at least one month before the patient receives the NK cell and/or NK-like T cell therapy.

In one embodiment the patient is administered the anti-viral agent one day before the patient receives the NK cell and/or NK cell therapy.

Preferably, the anti-viral agent is administered at least one day after the patient received the NK cell and/or NK-like T cell therapy, such as: at least two days after; or at least three days after; or at least four days after the patient received the NK cell and/or NK-like T cell therapy.

In an embodiment, the anti-viral agent is administered at least one day after the patient received the NK cell and/or NK-like T cell therapy, such as: at least two days after; or at least three days after; or at least four days after; or at least five days after; or at least six days after; or at least one week after; or at least two weeks after; or at least three weeks after; or at least one month after the patient received the NK cell and/or NK-like T cell therapy.

The dose or amount of the anti-viral agent administered to the patient should be a therapeutically effective amount for the intended purpose, i.e., prevention and/or prophylaxis and/or in amount effective to kill or inactivate the virus.

In one embodiment, the anti-viral agent is administered at a suitable dose ranging from about 1 to about 100 mg/kg of body weight per day, preferably within the range of about 2 to 50 mg/kg/day, most preferably in the range of 3 to 20 mg/kg/day. The desired dose may conveniently be presented in a single dose or as divided doses administered at appropriate intervals, for example as two, three, four or more sub-doses per day. In one embodiment, 500-1500 mg of the anti-viral agent is administered to the patient within a 24 hour period. In other words, in any given day the patient will receive 500-1500 mg of the anti-viral agent.

The particular, therapeutically-effective dose for a particular patient will depend on a variety of factors including the disorder being treated and the severity of the disorder; activity of the specific agent(s) employed; the age, body weight, general health, sex and diet of the patient; the time of administration; the route of administration; the rate of excretion of the specific agent(s) employed; the duration of the treatment; drugs used in combination or coincidental with the specific agent(s) employed and like factors well known in the medical field. For example, it is well within the skill of the art to start doses of the agent at levels lower than those required to achieve the desired therapeutic effect and to gradually increase the dosage until the desired effect is achieved. If desired, the effective daily doses may be divided into multiple doses for purposes of administration. Consequently, single dose compositions may contain such amounts or submultiples to make up the daily dose.

Accordingly, in one embodiment, the anti-viral agent is administered to the patient in one or more dose. In an embodiment the anti-viral is administered to the patient in one, two, three, four, five or six doses. In a preferred embodiment the anti-viral agent is administered to the patient in two doses. In other words, the patient receives the ant-viral agent twice daily.

Preferably, the anti-viral agent is administered to the patient in a dose of 250-1500 mg in a 24 hour period, such as: 250 mg in a 24 hour period; or 300 mg in a 24 hour period; or 400 mg in a 24 hour period; or 500 mg in a 24 hour period; or 600 mg in a 24 hour period; or 700 mg in a 24 hour period; or 800 mg in a 24 hour period; or 900 mg in a 24 hour period; or 1000 mg in a 24 hour period; or 1100 mg in a 24 hour period; or 1200 mg in a 24 hour period; or 1300 mg in a 24 hour period; or 1400 mg in a 24 hour period; or 1500 mg in a 24 hour period.

Preferably, the anti-viral agent is administered to the patient in two doses of 250-750 mg in a 24 hour period, preferably wherein the anti-viral agent is administered to the patient in two doses of 500 mg in a 24 hour period.

It will be appreciated that administration of the anti-viral agent can occur as a single event or over a time course of treatment. For example, one or more of the anti-viral agents can be administered hourly (e.g., every hour, every two hours, every three hours, every four hours, every five hours, every six hours, and so on), daily, twice daily, weekly, bi-weekly, or monthly. Certain conditions could extend prophylaxis from several days to several weeks. For example, prophylaxis could extend over one week, two weeks, or three weeks, or prophylaxis could extend from several weeks to several months.

Accordingly, in one embodiment, the anti-viral agent is administered to the patient for a duration of at least one month, such as: at least two months; or at least three months; or at least four months; or at least five months; or at least six months; or at least seven months. In a further embodiment, the anti-viral agent is administered for the patient for a duration of up to 100 days. In a preferred embodiment, the anti-viral agent is administered for the patient for a duration of up to seven months, such as six months. In a further preferred embodiment, 500 mg of the anti-viral agent is administered to the patient twice in 24 hours, for a duration of six months.

It will be appreciated that in some cases the anti-viral agent is administered for a duration of longer than seven months, such as eight or nine months.

In one embodiment, the patient receives NK cell and/or NK-like T cell therapy following high dose therapy (HDT) and/or autologous stem cell transplantation (ASCT).

By “high dose therapy (HDT)” we include high dose chemotherapy, also called “intensive therapy”. In a patient with multiple myeloma HDT includes chemotherapy with melphalan (brand name: Alkeran), cyclophosphamide (brand name: Cytoxan), doxorubicin (brand name: Adriamycin), liposomal doxorubicin (brand name: Doxil), and/or panobinostat (brand name: Farydak). HDT may include multiple rounds of chemotherapy.

By “autologous stem cell transplantation (ASCT)” we include a transplantation comprising stem cells obtained from the patient's own blood or bone marrow.

In an alternative embodiment, the stem cell transplant is an allogeneic transplantation, wherein the stem cells or bone marrow are obtained from a donor with a matching tissue type (for example, a close relative). In an alternative embodiment, the stem cell transplant is a syngeneic transplantation, wherein the stem cells or bone marrow are obtained from an identical twin.

Preferably the stem cell transplantation is autologous (ASCT) (Gertz, M. A., & Dingli, D. (2014). How we manage autologous stem cell transplantation for patients with multiple myeloma. Blood, 124(6), 882-890. Accessed Mar. 25, 2018).

High-dose chemotherapy followed by autologous peripheral blood stem cell transplantation (ASCT) is currently a standard treatment approach for patients with multiple myeloma aged 65 years and under.

In a further embodiment, the patient receives NK cell and/or NK-like T cell therapy between three and seven months after ASCT, such as three months, four months, five months, six months, or seven months after ASCT. Preferably, the patient receives NK cell and/or NK-like T cell therapy six months after ASCT.

In one embodiment, the NK cell and/or NK-like T cell therapy comprises one, or two, or three, or four, or five administrations of NK cells and/or NK-like T. Preferably, the patient receives three administrations of NK cells and/or NK-like T cells.

In a further embodiment, the NK cells and/or NK-like T cells are administered at a dosage of between 5×10⁶ to 100×10⁶cells/kg body weight of the patient, for example: at least 5×10⁶ cells/kg body weight of the patient; or at least 50×10⁶ cells/kg body weight of the patient; or at least 100×10⁶ cells/kg body weight of the patient. In a preferred embodiment, the patient receives three administrations of the NK cells and/or NK-like T cells at escalating doses of 5×10⁶, 50×10⁶, and up to 100×10⁶ cells/kg body weight within an interval of one week.

As discussed above, anti-viral agents that are applicable in the context of the present invention are those which target any of the stages of the life cycle of a virus, selected from one or more of: attachment to a host cell; release of viral genes and/or enzymes into the host cell; replication of viral components using host-cell machinery; assembly of viral components into complete viral particles; and release of viral particles to infect new host cells.

Typical anti-viral medications used against herpes viruses work by interfering with viral replication, effectively slowing the replication rate of the virus and providing a greater opportunity for the immune response to intervene.

Anti-viral agents can be selected from the group comprising: agents acting on viral DNA polymerase, such as nucleoside analogues after phosphorylation to their triphosphate forms and phosphonoformic and phosphonoacetic acids and their analogues.

In some embodiments, the anti-viral agent is an anti-viral agent selected from the group comprising: valomaciclovir stearate (EPB-348), octadecyloxyethyl-cidofovir (ODE-CDV, CMX-001), hexadecyloxypropyl-cidofovir (HDP-CDV), abacavir, adefovir, amantadine, amprenavir, arbidol, atzanavir, atripla, combivir, darunavir, delavirdine, didanosine, docosanol, edoxudine, efavirenz, emtricitabine, enfuvirtide, entecavir, entry inhibitors, antiretroviral, fomivirsen, fosamprenavir, fusion inhibitors, gardasil, ibacitabine, imunovir, idoxuridine, imiquimod, indinavir, inosine, integrase inhibitor, interferon type III, interferon type II, interferon type I, interferon, lamivudine, lopinavir, lopinavir, loviride, MK-0518, maraviroc, moroxydine, nelfinavir, nevirapine, nexavir, nucleoside analogues, oseltamivir, peramivir, pleconaril, podophyllotoxin, protease inhibitors, reverse transcriptase inhibitors, ribavirin, rimantadine, ritonavir, saquinavir, stavudine, synergistic enhancer, tenofovir, tenofovir disproxil, tipranavir, trifluridine, trizivir, tromantadine, truvada, valavivlovir, vicriviroc, vidarabine, viramidine, zalcitabine, zanamivir, zidovudine, A-5021 ([1′S,2′R)-9[[1′2′-bis(hydroxymethyl)cycloprop-1′-yl]-methyl]guanine]), cyclopropavir (CPV, ZSM-I-62), 2,4-diamino-6-R43-hydroxy-2(phosphonomethoxy)propoxy]-pyrimidine (HPMPO-DaPy), N-(4-chlorobenzyl)-1-methyl-6-(4-morpholinylmethyl)-4-oxo-1,4-dihydro-3-quinolinecarboxamide (PNU-183792), 2-bromo-5,6-dichloro-1-(beta-D-ribofuranosyl)benzimidazole (BDCRB), 1-(beta-L-ribofuranosyl)-2-isopropylamino-5,6-dichlorobenzimidazole (Maribavir, 1263W94), 3-hydroxy-2,2-dimethyl-N[4-{[(5-dimethylamino)-1-naphthylj-sulfonyl}-amino)phenyl}propamide (BAY 38-4766), 4-(2-amino-4-thiazolyl)phenyl derivative (BILS 179BS), N45-(aminosulfonyl)-4-methyl-1,3-thiazol-2-yl]-N-methyl-2-{4-(2-pyridinyl)phenyl}acetamide (BAY 57-1293), 2H-3-(4-chlorophenyl)-3,4-dihydro-1,4-benzo-thiazine-2-carbonitrile-1-oxide or 1,1-dioxide and 2-chloro-3-pyridin-3-yl-5,6,7,8-tetrahydronindolizine-1-carboxamide (CMV423).

Accordingly, in one embodiment the anti-viral agent comprises a nucleoside analogue, such as one selected from the group consisting of: valacyclovir; acyclovir; famciclovir; and/or penciclovir or an active metabolite, prodrug, salt, solvate or hydrate of such nucleoside analogues. Other nucleoside analogues with anti-viral activity may be identified by standard methods known in the art. In one embodiment the anti-viral agent is selected from the group comprising: valacyclovir or a prodrug or salt thereof; acyclovir or a prodrug or salt thereof; famciclovir or a prodrug or salt thereof and/or penciclovir or a prodrug or salt thereof.

It will be appreciated that the anti-viral agent of the invention may comprise a mixture of one or more anti-viral agents. In a preferred embodiment, 500 mg of valacyclovir is administered to the patient in two doses within a 24 hour period for a duration of six months prior to and during NK cell and/or NK-like T cell therapy.

As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a patient, but is then converted in vivo to an active compound, for example, by hydrolysis (e.g., hydrolysis in blood or a tissue). In certain cases, a prodrug has improved physical and/or delivery properties over a parent compound from which the prodrug has been derived. The prodrug often offers advantages of solubility, tissue compatibility, or delayed release in a mammalian organism.

Acyclovir and penciclovir are both guanosine analogues that inhibit viral replication by acting as a substrate for viral DNA polymerase. Valacyclovir is a prodrug, an esterified version of acyclovir that has greater oral bioavailability. Famciclovir is a prodrug form of penciclovir with improved oral bioavailability. The following anti-viral agents are all analogues of acyclic guanosine and are commercially available: Zovirax® (acyclovir), Valtrex® (valacyclovir), Denavir® (penciclovir), and Famvir® (famciclovir).

Other nucleoside analogues with anti-viral activity are known in the art and may be used in the context of the present invention. For example, Bromovinyl deoxyuridine (Brivudin) is a highly potent thymidine nucleoside analogue with selective activity against HSV-1 and VZV. It is understood that bicyclic pyrimidine nucleoside analogues (BCNAs) have anti-viral activity.

Anti-viral agents for use in the context of the present invention also include vaccines. Zostavax® is a live, attenuated varicella-zoster vaccine used to prevent shingles and zoster-related post-herpetic neuralgia (PHN), the long-lasting nerve pain that follows shingles. The vaccine can be injected subcutaneously (SC) or intramuscularly (IM). Shingrix® is a non-live, recombinant subunit varicella-zoster vaccine used to prevent shingles (zoster) and reduce the overall incidence of PHN. It combines an antigen, glycoprotein E, and an adjuvant system. The vaccine can be injected intramuscularly (IM).

It will be appreciated by those skilled in the art that the anti-viral agents of the present invention may also be utilized in the form of a pharmaceutically acceptable salt or solvate thereof. The pharmaceutically acceptable salts of the anti-viral agent include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

The anti-viral agent may be administered by parenteral administration. By parenteral administration we include any non-oral means of administration, such as injecting directly into the body, bypassing the skin and mucous membranes. The common parenteral routes are intramuscular (IM), subcutaneous (SC) and intravenous (IV).

In one embodiment of the invention, the anti-viral agent is administered orally, intravenously, subcutaneously, intravenously and/or intramuscularly.

In one embodiment a herpes virus selected from the group comprising: varicella zoster virus (VZV); herpes simplex virus (HSV); Epstein Barr Virus (EBV); and/or cytomegalovirus (CMV), is present in the patient. In one embodiment, the herpes virus us dormant (latent). It will be appreciated that this patient may be termed “seropositive”.

By “seropositive” we include the presence of antibodies or other immune markers in serum from a patient, that indicate prior exposure to a particular organism, antigen or virus. It will be appreciated that the patient may be seropositive for one or more of the herpes virus selected from the group comprising: varicella zoster virus (VZV); herpes simplex virus (HSV); Epstein Barr Virus (EBV); and cytomegalovirus (CMV) before receiving a therapy comprising natural killer (NK) cells and/or NK-like T cells. Methods for determining the presence of a latent virus in a patient are known in the art, for example PCR, such as RT-PCR.

In one embodiment the herpes virus infection causes shingles.

It will be appreciated that the anti-viral agent could be used to prevent shingles in a patient who has received a therapy comprising natural killer (NK) cells and/or NK-like T cells. It will be understood that the anti-viral agent could also be used to prevent serious complications of shingles such as zoster-related post-herpetic neuralgia (PHN), zoster multiplex, myelitis, herpes ophthalmicus, or zoster sine herpete, in a patient who has received a therapy comprising natural killer (NK) cells and/or NK-like T cells.

In a fourth aspect, the invention provides natural killer (NK) cells and/or NK-like T cells for use in treating a malignant disease in a patient, wherein the use comprises the step of administering an anti-viral agent to the patient with the NK cell and/or NK-like T cell therapy.

In a fifth aspect, the invention provides use of natural killer (NK) cells and/or NK-like T cells in the manufacture of a medicament for treating a malignant disease in a patient, wherein the patient receives an anti-viral agent with the NK cell and/or NK-like T cell therapy.

In a sixth aspect, the invention provides a method for treating a malignant disease in a patient, comprising the step of administering a therapy comprising natural killer (NK) cells and/or NK-like T cells, and further comprising the step of administering an anti-viral agent to the patient with the NK cell and/or NK-like T cell therapy.

In an embodiment of the invention, the anti-viral agent prevents a herpes virus infection in a patient. In an embodiment of the invention, the herpes virus infection comprises reactivation of the herpes virus. It will be appreciated that the herpes virus may include those described above in relation to the first, second and third aspects of the invention.

In an embodiment of the invention, the NK cell and/or NK-like T cell therapy induces and/or increases herpes virus reactivation. It will be appreciated that the NK cell and/or NK-like T cell therapy include those described above in relation to the first, second and third aspects of the invention.

In an embodiment of the invention, the patient receives NK cell and/or NK-like T cell therapy following high dose therapy (HDT) and/or autologous stem cell transplantation (ASCT).

In an embodiment of the invention, the anti-viral agent is as defined above in the context of the first, second and third aspects of the invention.

In a seventh aspect, the invention provides a pharmaceutical composition comprising: NK cells and/or NK-like T cells and an anti-viral agent as defined above in the context of the first, second and third aspects of the invention.

In an embodiment, the pharmaceutical composition further comprises a pharmaceutically acceptable diluent, carrier or excipient. By “pharmaceutically acceptable” is included that the composition or formulation is sterile and pyrogen free. Suitable pharmaceutical carriers, diluents and excipients are well known in the art of pharmacy. The carrier(s) must be “acceptable” in the sense of being compatible with the inhibitor and not deleterious to the recipients thereof. Typically, the carriers will be water or saline which will be sterile and pyrogen free; however, other acceptable carriers may be used.

It will be appreciated that the pharmaceutical composition comprises a therapeutically effective amount of the anti-viral agent for the intended purpose, i.e., prevention or prophylaxis and/or in amount effective to kill or inactivate the virus.

In an embodiment of the invention the pharmaceutical composition is for use in preventing a herpes virus infection in a patient, wherein the patient has received a therapy comprising natural killer (NK) cells and/or NK-like T cells, wherein the use comprises the step of administering the pharmaceutical composition to the patient with the NK cell and/or NK-like T cell therapy.

In a further embodiment the invention provides use of the pharmaceutical composition in the manufacture of a medicament for preventing a herpes virus infection in a patient, wherein the patient has received a therapy comprising natural killer (NK) cells and/or NK-like T cells, wherein the pharmaceutical composition is administered to the patient with the NK cell and/or NK-like T cell therapy.

In an embodiment, the invention provides a method for preventing a herpes virus infection in a patient, wherein the patient has received a therapy comprising natural killer (NK) cells and/or NK-like T cells, wherein the method comprises the step of administering the pharmaceutical composition to the patient with the NK cell and/or NK-like T cell therapy.

The following are pharmaceutical formulations and/or compositions according to the invention in which the active ingredient is an anti-viral agent as defined herein.

The formulations or compositions include those suitable for oral and parenteral (including subcutaneous e.g. by injection or by depot tablet, intradermal, intrathecal, intramuscular e.g. by depot and intravenous) administration although the most suitable route may depend upon for example the condition, age, and disorder of the recipient as well as the viral infection or disease being treated.

In an embodiment, the pharmaceutical compositions or formulations of the invention are for parenteral administration, more particularly for intravenous or subcutaneous administration. In a preferred embodiment, the pharmaceutical composition is suitable for intravenous or subcutaneous administration to a patient, for example by injection.

Formulations or compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Preferably, the formulation is a unit dosage containing a daily dose or unit, daily sub-dose or an appropriate fraction thereof, of the active ingredient.

The agent or active ingredient may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the agent or active ingredient will generally be administered in admixture with a suitable pharmaceutical excipient, diluent or carrier selected with regard to the intended route of administration and standard pharmaceutical practice.

For example, the agent or active ingredient may be administered orally, buccally or sublingually in the form of tablets, capsules, ovules, elixirs, solutions or suspensions, which may contain flavouring or colouring agents, for immediate-, delayed- or controlled-release applications. The active ingredient may also be administered via intracavernosal injection.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The agent or active ingredient can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of an agent, antibody or compound will usually be from 1 to 1,000 mg per adult (i.e. from about 0.015 to 15 mg/kg), administered in single or divided doses.

Thus, for example, the tablets or capsules of the agent or active ingredient may contain from 1 mg to 1,000 mg of agent or active agent for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The agent or active ingredient can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an active ingredient and a suitable powder base such as lactose or starch. Such formulations may be particularly useful for treating solid tumours of the lung, such as, for example, small cell lung carcinoma, non-small cell lung carcinoma, pleuropulmonary blastoma or carcinoid tumour.

Aerosol or dry powder formulations are preferably arranged so that each metered dose or “puff” contains at least 1 mg of the inhibitor for delivery to the patient. It will be appreciated that the overall daily dose with an aerosol will vary from patient to patient, and may be administered in a single dose or, more usually, in divided doses throughout the day.

Alternatively, the agent or active ingredient can be administered in the form of a suppository or pessary, particularly for treating or targeting colon, rectal or prostate tumours.

In an embodiment, the agent or active ingredient may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The agent or active ingredient can be administered by a surgically implanted device that releases the drug directly to the required site, for example, into the eye to treat ocular tumours. Such direct application to the site of disease achieves effective therapy without significant systemic side-effects.

An alternative method for delivery of agents or active ingredients is the Regel injectable system that is thermo-sensitive. Below body temperature, Regel is an injectable liquid while at body temperature it immediately forms a gel reservoir that slowly erodes and dissolves into known, safe, biodegradable polymers. The active drug is delivered over time as the biopolymers dissolve.

Polypeptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Polynucleotides may be administered as a suitable genetic construct as described below and delivered to the patient where it is expressed. Typically, the polynucleotide in the genetic construct is operatively linked to a promoter which can express the compound in the cell. The genetic constructs of the invention can be prepared using methods well known in the art, for example in Sambrook et al (2001).

Although genetic constructs for delivery of polynucleotides can be DNA or RNA, it is preferred if they are DNA.

Preferably, the genetic construct is adapted for delivery to a human cell. Means and methods of introducing a genetic construct into a cell are known in the art, and include the use of immunoliposomes, liposomes, viral vectors (including vaccinia, modified vaccinia, lentivurus, parvovirus, retroviruses, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art. In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Although for cancer/tumours of specific tissues it may be useful to use tissue-specific promoters in the vectors encoding a polynucleotide inhibitor, this is not essential, as the risk of expression of the active ingredient in the body at locations other than the cancer/tumour would be expected to be tolerable in compared to the therapeutic benefit to a patient suffering from a cancer/tumour. It may be desirable to be able to temporally regulate expression of the polynucleotide inhibitor in the cell, although this is also not essential.

The agents or active ingredients of the invention (i.e. an anti-viral agent) may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of protein activity loss and that use levels may have to be adjusted upward to compensate. In one embodiment, the lyophilised (freeze dried) active ingredient loses no more than about 20%, or no more than about 25%, or no more than about 30%, or no more than about 35%, or no more than about 40%, or no more than about 45%, or no more than about 50% of its activity (prior to lyophilisation) when re-hydrated.

It will be appreciated that the amount of an anti-viral agent required for use in prophylaxis will vary with the nature of the condition being treated and the age and the condition of the patient and will be ultimately at the discretion of the attendant physician. In general, however, doses employed for adult human treatment will typically be in the range of 0.02-5000 mg per day, preferably 100-1500 mg per day. The desired dose may conveniently be presented in a single dose or as divided doses administered at appropriate intervals, for example as two, three, four or more sub-doses per day. The formulations according to the invention may contain between 0.1-99% of the active ingredient, conveniently from 30-95% for tablets and capsules and 3-50% for liquid preparations.

Of course, one of ordinary skill in the art may modify the formulations within the teachings of the specification to provide numerous formulations for a particular route of administration without rendering the compositions of the present invention unstable or compromising their therapeutic activity. It is also well within the ability of the skilled person to modify the route of administration and dosage regimen of a particular compound in order to manage the pharmacokinetics of the present compounds for maximum beneficial effect to the patient.

In an eighth aspect, the invention provides a kit of parts comprising:
(i) a composition comprising NK cells and/or NK-like T cells, wherein the NK cells and/or NK-like T cells are as defined herein; and
(ii) an anti-viral agent as defined herein.

In an embodiment, the kit further comprises a pharmaceutically acceptable diluent, carrier or excipient, such as those described above in the context of the pharmaceutical composition. In a further embodiment, the kit further comprises instructions for administering the NK cells and/or NK-like T cells and/or the anti-viral agent to a patient.

In an embodiment, and as described above in the context of the first, second and third aspects of the invention, the anti-viral agent is for use in preventing a herpes virus reactivation in a patient.

In a preferred embodiment, and as described above in the context of the first, second and third aspects of the invention, the herpes virus infection comprises reactivation of the herpes virus.

In an embodiment, and as described above in the context of the first, second and third aspects of the invention, the NK cell and/or NK-like T cell therapy induces and/or increases herpes virus reactivation.

In an embodiment of the invention the kit of parts is for use in preventing a herpes virus infection in a patient, wherein the patient has received a therapy comprising natural killer (NK) cells and/or NK-like T cells, wherein the use comprises the step of administering the components of the kit of parts to the patient with the NK cell and/or NK-like T cell therapy.

In a further embodiment the invention provides use of the kit of parts in the manufacture of a medicament for preventing a herpes virus infection in a patient, wherein the patient has received a therapy comprising natural killer (NK) cells and/or NK-like T cells, wherein the components of the kit of parts are administered to the patient with the NK cell and/or NK-like T cell therapy.

In an embodiment, the invention provides a method for preventing a herpes virus infection in a patient, wherein the patient has received a therapy comprising natural killer (NK) cells and/or NK-like T cells, wherein the method comprises the step of administering the components of the kit of parts to the patient with the NK cell and/or NK-like T cell therapy.

It will be appreciated that, the features of the fourth, fifth, sixth, seventh and eighth aspects of the invention may be as described herein in relation to the other aspects of the invention.

All of the documents referred to herein are incorporated herein, in their entirety, by reference.

The listing or discussion of an apparently prior published document in this specification should not necessarily be taken as an acknowledgement that the document is part of the state of the art or is common general knowledge.

The invention will now be described by reference to the following Figures and Examples.

Preferred, non-limiting examples which embody certain aspects of the invention will now be described, with reference to the following figures:

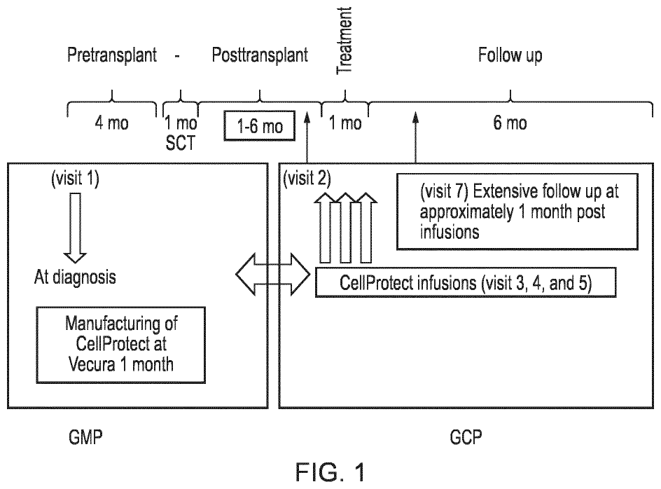

FIG. 1: “CellProtect” Safety Study

Schematic chart: A safety study of “CellProtect”, an autologous ex vivo expanded and activated NK cell product, in patients with Multiple Myeloma (ACP-001).

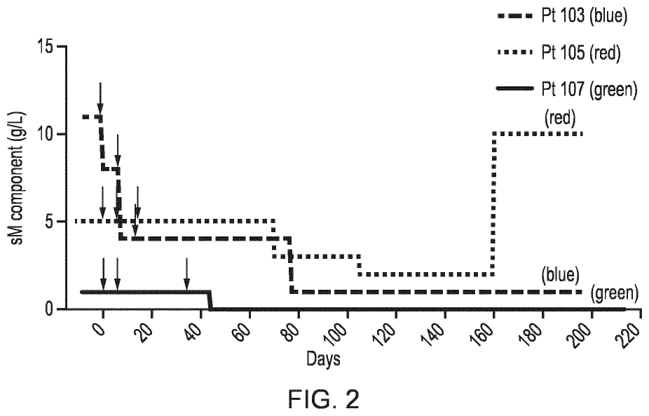

FIG. 2: Clinical efficacy of the “CellProtect” treatment

Six patients have been infused with three doses of “CellProtect” (indicated by arrows). Two patients had a measurable M component at time of infusion. Both of these patients responded with at least 50% reduction in serum M component (biomarker for tumour burden). This reduction is considered clinical “response” (Pt 103) and (Pt 105). Five months' post infusion, one of these patients relapsed (Pt 105). One patient with detectable M component showed an improved response post CellProtect infusion (Pt 107). The other three patients did not show any amounts of M component before infusion (not shown). None of the patients in complete remission (CR) relapsed during the course of treatment and clinical follow up.

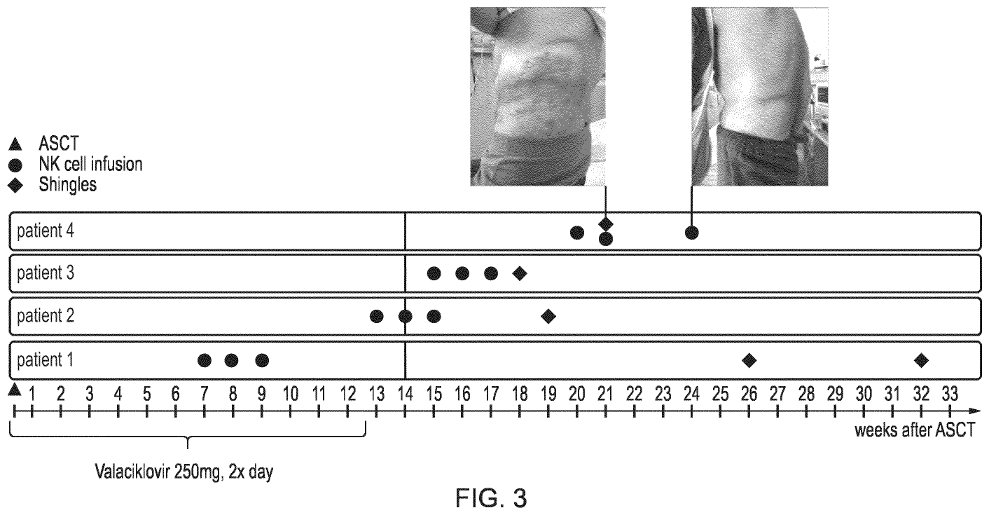

FIG. 3: “CellProtect” infusion and reactivation timeline

Summary of the infusion and reactivation timelines in the context of prior ASCT, of the first four patients. “Patient 1” is Patient 103, “Patient 2” is Patient 105, “Patient 3” is Patient 106 and “Patient 4” is Patient 107.

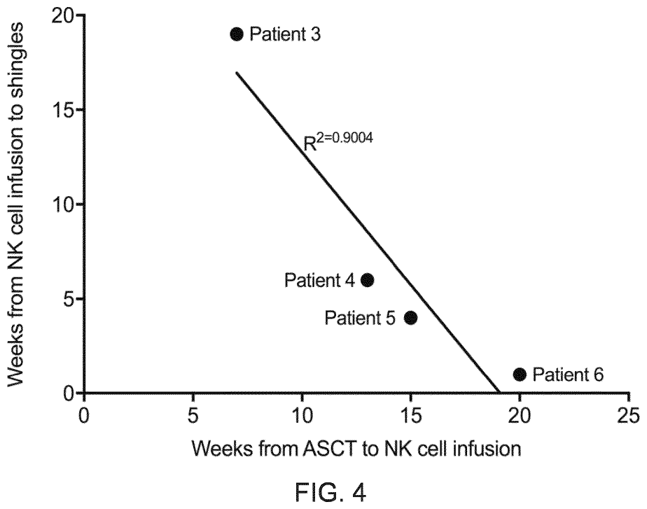

FIG. 4: Correlation between the time from ASCT to the NK cell infusion and the time from NK cell infusion to the development of HZ

Graph depicting the correlation between the time from ASCT to the NK cell infusion and the time from NK cell infusion to the development of HZ. “Patient 3” is Patient 103, “Patient 4” is Patient 105, “Patient 5” is Patient 106 and “Patient 6” is Patient 107.

FIG. 5: Manufacturing Process of “CellProtect” Drug Substance (DS) and Drug Product (DP)

Flow Chart depicting the manufacturing process of CellProtect.

### EXAMPLE 1

### Shingles Manifestation After Autologous Ex Vivo Expanded NK Cell Infusions in Patients with Multiple Myeloma (MM): the Need for Antiviral Prophylaxis

Introduction

The development of new drugs for treatment of multiple myeloma (MM) has improved survival significantly from previously about 3 years to now more the 5 years. However despite this dramatic improvement cure is practically never obtained, with the possible exception of a fraction of young patients treated with allogeneic stem cell transplantation (AlloSCT). New approaches are therefore important.

MM is a malignant neoplasm characterized by clonal proliferation of plasma cells in the bone marrow (BM). It is considered incurable due to persistence of minimal residual disease despite both novel and intensive treatment (1). We have previously shown that long-term ex vivo expanded and activated autologous NK cells from MM patients provide cytotoxic activity against autologous myeloma cells in vitro which is superior to that of short-term activated autologous NK cells (2). We have also shown that such cells can be used as efficient treatment of MM in experimental animals (3).

Recently we have optimized the procedure for NK cell expansion in a close-automated bioreactor using clinical grade GMP-compliant components (4), finalized all preclinical requirements, obtained approval from the Swedish Medicinal Products Agency (EudraCT: 2010-022330-83) and the ethical committees (EPN: 2013/490-32) to initiate a first-in-man Phase I/II clinical trial. The expanded cells are fully compliant with the new EU ATMP Directives.

The aim with the present Phase I/II study of patients treated upfront with autologous stem cell transplantation (ASCT) was primarily to investigate the safety of expanded activated autologous NK cells and secondly to analyze efficacy parameters such as monoclonal immunoglobulins, response according to International Myeloma Working Group (IMWG) criteria and minimal residual disease following NK cell treatment of patients responding to ASCT.

The Advanced Therapy Medicinal Product (ATMP) CellProtect, is a cell suspension based on ex vivo expanded polyclonal NK cells with restored cytotoxic activity. The product is individually prepared and the treatment is autologous.

A first in human, phase I/II, therapeutic exploratory clinical trial with CellProtect in newly diagnosed patients with multiple myeloma was initiated 2014 at the Department of Hematology Karolinska University Hospital, Huddinge (EudraCT No 2010-022330-83), (FIG. 1). The key patient inclusion criteria are; MM, diagnosed according to Greipp P R, San Miguel J, Dune B G, et al. (2005), Eligible for, and willing to undergo, high dose chemotherapy and ASCT and Eastern Cooperative Oncology Group (ECOG) performance status 0-2.

The clinical study is an open, single arm, triple escalating dose/patient study to primarily investigate the safety and tolerability of CellProtect in patients with MM following ASCT. The secondary objectives are to investigate the effect of CellProtect to deepening the response, i.e. further decrease in M-protein in patients who did not achieve complete remission or reaching a less minimal residual disease (MRD) in patients achieving complete remission.

Results

Six patients have completed the study including evaluation and a six month follow-up after last infusion (FIG. 1). A per protocol interim analysis of the clinical results is being finalized with an option to close the study, as safety data judged sufficient, alternatively keep it open and include additional six patients.

After being included in the study, the study patients will first donate blood for the production of CellProtect. The starting material is collected through a regular blood donation and transferred to the production facility where the finished product CellProtect is manufactured. CellProtect is stored cryopreserved at −150° C. at doses tailored to the patients until requested by referral from the Principal Investigator. The applicable bag(s) is transported by the Manufacturer on liquid nitrogen to the clinic where it is thawed shortly before the infusion.

The shelf life for active substance is based on an ongoing stability monitoring program from validation batches of CellProtect. Based on these data it is concluded that CellProtect Drug Product (DP) is stable at —150 ° C. for up to 48 months. Further data will be collected and it is expected that shelf life for the finished product can be extended. The in-use stability for the active substance has been determined to at least 60 minutes.

Subsequent to the blood donation, the patients are treated according to current clinical praxis with 3-4 cycles Cyber-D (Cyclophosphamide, Bortezomib, Dexamethasone) as induction followed by high dose treatment (Melphalan 200 mg/m2) and stem cell infusion.

The study treatment is initiated when the patients have recovered from the ASCT but within six months from the ASCT. The patients received three infusions of CellProtect at escalating doses of 5×10⁶; 50×10⁶; and up to 100×10⁶ cells/kg body weight with an interval of one week. The dose escalation is within each patient. The CellProtect treatment was assessed during a 6 month follow up period after the last infusion. During this follow up period the effect of the treatment was evaluated in more depth at study visit seven (7), approximately one month after the third CellProtect infusion. At visit 7, separate blood samples and bone marrow material have been collected for exploratory analyses of specific immunogenic response to the CellProtect treatment at this time point. These samples will be analysed with designed methods to specifically assess and explore the mechanisms of action of the Cell Protect Investigational Medicinal Product (IMP).

Clinical Results

Status of the Clinical Study

Six (6) patients have completed a valid per protocol CellProtect treatment, with three infusions with the per protocol requested accumulated number of activated NK cells. These 6 patients have been evaluated at visit seven (7) and subsequently completed the six months follow-up.

Patient Population Studied

The patients are included in the clinical trial at diagnosis, the patient demographic data are shown in Table 1. Five patients had IgG myeloma (103,105,106,107,111) and one IgA myeloma (110). The response status following ASCT and before NK cell infusion was very good partial response (VGPR) in three patients (103,105,107) and complete response (CR) in three (106,110,111). The starting material for the manufacturing of CellProtect is collected by a blood donation at the first (1) study visit, prior to initiation of any MM treatment.

TABLE 1
Patient Characteristics Study patient demographic data.

| Pat No | Sex | Age/yrs | Weight/kg | ECOG | ISS |
|---|---|---|---|---|---|
| 103 | F | 6 | 59 | 1 | III |
| 105 | F | 66 | 61 | 0 | II |
| 106 | M | 57 | 87 | 0 | II |
| 107 | M | 73 | 63 | 1 | III |
| 110 | M | 61 | 92 | 1 | II |
| 111 | M | 66 | 68 | 1 | I |

Response to the High Dose Chemotherapy and the ASCT

The second (2) study visit is a check -up visit after the ASCT, before the infusion of CellProtect. The purpose of this visit is to establish the patient's categorization according to the MM response criteria, Table 2, and to collect information about the patient's physical condition in order to confirm that the patient is still eligible and well enough to continue to study treatment. A bone marrow sample should be taken at this visit, provided consent from the patient and constitutes the baseline BM sampling for the assessment of the CellProtect treatment. The patient's status pre-dose at Visit 3 serves as baseline for most other assessments.

TABLE 2
Response at the check-up visit after HDC and ASCT. CR is defined as 0
in M-protein and less than 5% plasma cells in bone marrow aspirate

| | Visit 2 | |
|---|---|---|
| | Days after | |
| Pat No | transplant | Response |
| 103 | 41 | VGPR |
| 105 | 78 | VGPR |
| 106 | 97 | CR |
| 107 | 172 | VGPR/CR |
| 110 | 92 | CR |
| 111 | 111 | CR |

complete response (CR), very good partial response (VGPR)

Preliminary Clinical Results

Patient 103 was categorized to very good partial response (VGPR) to the ASCT treatment and was infused with three complete doses of CellProtect, 6-8 weeks after the transplantation. A reduction in M components serum levels from 8 g/L before to 1 g/L (>80% reduction) after the CellProtect infusion and it remained low over the study period.

Patient 105 was categorized to very good partial response (VGPR) to the ASCT treatment and was infused with three complete doses of CellProtect, 12-14 weeks after the transplantation. A reduction in M components serum levels was measured from 5 g/L to 2 g/L (app 60%) after CellProtect infusions and remained low for 4 months. The patients relapsed five months after the CellProtect infusions. Patient 105 did not consent to and therefore no bone marrow sampling was taken at visit 2.

Patient 106 was categorized to complete response (CR) to the ASCT treatment and was infused with three doses of CellProtect, 15-17 weeks after the transplantation. The third dose was reduced due to scarcity of the IMP. The patient remained in complete remission (CR) over the clinical follow up.

Patient 107 was first categorized to very good partial response (VGPR) and later to a confirmed complete remission (CR) to the ASCT treatment. Patient 107 had detectable levels of M component at the time for CellProtect infusions and was infused with three complete doses of CellProtect, 15-30 weeks after the transplantation. The third and highest dose of CellProtect was delayed due to activation of Herpes Zoster and manifestation of Shingles in the patient. Patient 107 showed an improved response post CellProtect infusions. A reduction in M components serum levels was measured from 1 g/L to 0 g/L. The free light chains (FLC) quota in this patient (736 at screening) was reduced from 1.6 to 0.8 during and after CellProtect infusions. The patient remained in complete remission (CR) over the clinical follow up.

Patient 110 was categorized to complete response (CR) to the ASCT treatment and was infused with three doses of CellProtect, 14-16 weeks after the transplantation. The third dose was reduced due to scarcity of the IMP. The patient remained in complete remission (CR) over the clinical follow up.

Patient 111 was categorized to complete response (CR) to the ASCT treatment and was infused with three complete doses of CellProtect, 17-19 weeks after the transplantation. The patient remained in complete remission (CR) over the clinical follow up.

TABLE 3
Response of the CellProtect treatment.

| | | | | | Best response | |
|---|---|---|---|---|---|---|
| | | | | | following CellProtect | |
| | Baseline | CellProtect infusions | | | infusions | |
| Pat No | M-component | Days after Transplant | | | M-component | Responder |
| 103 | 8 | 49 | 56 | 63 | 4 | Yes |
| 105 | 4 | 91 | 98 | 106 | 2 | Yes |
| 106 | 0 | 105 | 112 | 120 | 0 | NE* |
| 107 | 1 | 180 | 186 | 214 | 0 | Yes |
| 110 | 0 | 98 | 105 | 112 | 0 | NE* |
| 111 | 0 | 119 | 125 | 131 | 0 | NE* |

*NE = Not evaluable because of complete remission before administration of CellProtect

Efficacy

Signs of clinical efficacy of the CellProtect treatment have up to now been monitored by measuring the serum immunoglobulin levels, an established biomarker for the disease (FIG. 2). A reduction of the serum immunoglobulin levels is measured following administration of CellProtect in all (3) patients with remaining measurable disease, and were in stable partial response (VGPR) or (VGPR) after the ASCT. Three (3) of the six (6) patients were in complete remission (CR) and did not have measurable levels of the serum immunoglobulin at the CellProtect treatment. An increase of the immunoglobulin levels has up to now been measured in one of the six (6) patients treated with CellProtect (FIG. 2).

Interestingly, in this clinical trial an activation of Herpes Zoster and manifestation of Shingles was observed in the first four patients (103, 105, 106 and 107) dosed with CellProtect. Patient 110 and 111 were treated with high dose prophylaxis prior to the CellProtect infusions which prevented the virus activation. This side effect of the CellProtect product further confirms the in vivo biological activity of the cell preparations.

Safety

No serious side effects were seen. However the first four patients (103, 105, 106 and 107) developed Herpes Zoster (HZ, shingles) 18-32 weeks following ASCT and 3-25 weeks following the first NK cell infusion (FIG. 3). These patients had received valaciclovir 250 mg×2 daily for 14 weeks following ASCT as prevention of viral reactivation according to clinical praxis. The drug had then been withdrawn, thus did not cover the time when HZ developed after NK cell infusion.

Since the development of HZ was clearly associated with the NK cell infusions in the first four patients the last two patients (110 and 111) were treated with high dose valaciclovir, 500 mg twice daily for 6 months following the infusions. These two patients did not show any signs of HZ activation within 12 months following the prophylaxis treatment

The infusion and reactivation timelines in the context of prior ASCT, of the first four patients, is summarized in FIG. 3.

Discussion

Our results show that autologous expanded and activated NK cells are safe but induce HZ reactivation unless antiviral drugs are used for prevention.

It seems clear that the NK cell infusions are the cause of the HZ development. HZ always appears after the NK cell infusion and there was in inverse correlation between the time from ASCT to the NK cell infusion and the time from NK cell infusion to the development of HZ as seen in FIG. 4.

A possible mechanism for the HZ development is an induction of an immunological cascade response upon adoptive activated NK cell transfer. It is conceivable that activated NK cells upon adoptive cell transfer attack reservoir cells for HZV which, in turn, due to induced stress, might cause a viral reactivation.

It is unlikely that the ASCT per se is responsible for the HZV activation considering the time of HZV development related to the times of ASCT and NK cell infusions respectively. Also, recent reports showed only 1.0-14.4% HZV infections in ASCT treated MM patients without antiviral prophylaxis (8, 9), which is far from the likelihood that four out of four consecutive patients (100%) as in our study should develop shingles due to ASCT.

Our conclusion is that NK cell-based immunotherapy is feasible in MM, however, it should always be combined with prophylactic antiviral treatment.

Materials and Methods

Manufacture of CellProtect

The CellProtect drug product is a cell suspension based on ex vivo expanded NK cells from patients with MM. The treatment is autologous.

The protocol for making CellProtect is described below and depicted in FIG. 5.

Peripheral blood from patients with MM is collected through a blood donation. According to the collection method, one-unit (app 450m1) of whole blood is collected into a sterile polyvinylchloride (PVC) plastic transfer bag. (Terumo or Fenwal blood collection container). The collected blood is stored in room temperature (15-25° C.) and the manufacture process starts within 6 hours.

Lymphocytes are separated by density-based gradient using Ficoll-Paque. After a final washing step with PBS the cells are counted and adjusted to 0.5 to 1.0×10⁶ cells /mL in 800 to 1000 mL cell culture media supplemented with the following materials: 500 IU/mL interleukin 2 (IL-2, a cytokine that activates NK cells), 10 ng/mL Orthoclone OKT3 (muromonab-CD3, a CD-3 antibody that stimulates growth of T-cells), 5% (v/v) human serum (growth promoting) and 0.1% (v/v) pluronic F68 (detergent to reduce foaming). The cells are seeded in the bioreactor and cultivation is started.

The peripheral blood lymphocytes are expanded using a closed Wave bioreactor system (System 2/10 GE Healthcare) which controls temperature to 37° C. and 5% CO2. The cells are grown in a disposable cellbag 2 L (culture volume 1 L). The cell contact surface is an ethylene vinyl acetate (EVA)/low density polyethylene copolymer. The outer layers are made of proprietary composites that provide exceptional strength and extremely low gas permeability.

When the cell density reaches 3×10⁶ cells/mL, perfusion starts by feeding the culture with cell medium supplemented with material described above but without Orthoclone OKT3. Perfusion is controlled in shots of 50 mL cell culture medium including 500 IU/mL IL-2, 5% human serum and 0.1% pluronic F68. The cell concentration determines the volume of the shots.

The NK cell expansion phase is commenced when perfusion starts and continues for 14 to 16 days.

Cell number and viability is monitored on a regular basis during the culture period. Flow cytometry analysis is conducted to determine the percentage of NK cells. Activated NK cells are analysed by expression of the surrogate marker for cytotoxicity CD107a after triggering with the K562 cell line. Sterility, endotoxin and mycoplasma testing are also performed on samples taken after expansion during the manufacturing of CellProtect DP.

Specification of the CellProtect Investigational Medicinal Product (IMP)

Tables 4 and 5 below outline the specification of the CellProtect product.

TABLE 4
Components of the Investigational Medicinal Product

| Component | Description of component | Amount |
|---|---|---|
| CellProtect | Cytotoxic NK cells in a suspension | 20 × 10⁶-180 × 10⁶ |
| drug | of cells also containing T | cells/ml |
| substance | lymphocytes and NK-like T-cells | |
| Human | Freezing media | Up to 95% of total |
| plasma | | volume |
| DMSO | Cryoprotectant | 5% |

TABLE 5
Specification for the Routine Control of CellProtect Drug Substance

| Test | Approval limit |
|---|---|
| Total number of cells | ≥15500 × 10⁶ |
| Viability | ≥90% |
| NK cells (CD3-CD56+) | ≥10% |
| Activated NK cells | ≥30% of NK cells |
| Sterility | Sterile |
| Endotoxin | ≤0.5 EU/mL |
| Mycoplasma | Not detectable |

Composition of Clinical Batches

The composition of clinical batches of CellProtect is shown in Table 6.

TABLE 6
Composition of the clinical batches

| | | total | total cells/kg | | | | |
|---|---|---|---|---|---|---|---|
| | | number | body weight | | % | % | |
| Patient | | cells | administered | % | Activated | NK-like | % |
| no | dose | ×10⁶ | ×10⁶ | NK cells | NK cells | T cells | T cells |
| 103* | 1 | 285 | 5 | 14.1 | 47.0 | 15.6 | 69.2 |
| | 2 | 2850 | 50 | | | | |
| | 3 | 5700 | 100 | | | | |
| 105* | 1 | 315 | 5 | 13.0 | 71.0 | 14.9 | 64.9 |
| | 2 | 3150 | 50 | | | | |
| | 3 | 6300 | 100 | | | | |
| 106 | 1 | 435 | 5 | 30.6 | 63.0 | 21.8 | 43.1 |
| | 2 | 4350 | 50 | | | | |
| | 3 | 4525 | 52** | | | | |
| 107 | 1 | 363 | 5 | 30.1 | 57.0 | 25.3 | 43.1 |
| | 2 | 3630 | 50 | | | | |
| | 3 | 7260 | 100 | | | | |
| 110 | 1 | 457.5 | 5 | 40.2 | 35.0 | 15.8 | 33.9 |
| | 2 | 4575 | 50 | | | | |
| | 3 | 3678 | 40** | | | | |
| 111 | 1 | 341.5 | 5 | 22.5 | 47.0 | 6.7 | 61.5 |
| | 2 | 3415 | 50 | | | | |
| | 3 | 6830 | 100 | | | | |

*patient showing decline in M-component level
**deviant amount of cell in dose 3

### EXAMPLE 1 REFERENCES

1. Alici E, Bjorkstrand B, Treschow A, Aints A, Smith C I, Gahrton G, et al. Long-term follow-up of gene-marked CD34+ cells after autologous stem cell transplantation for multiple myeloma. Cancer Gene Ther. 2007; 14(3):227-32.

2. Alici E, Sutlu T, Bjorkstrand B, Gilljam M, Stellan B, Nahi H, et al. Autologous antitumor activity by NK cells expanded from myeloma patients using GMP-compliant components. Blood. 2008; 111(6):3155-62.

3. Alici E, Konstantinidis K V, Sutlu T, Aints A, Gahrton G, Ljunggren H G, et al. Anti-myeloma activity of endogenous and adoptively transferred activated natural killer cells in experimental multiple myeloma model. Exp Hematol. 2007; 35(12):1839-46.

4. Sutlu T, Stellan B, Gilljam M, Quezada H C, Nahi H, Gahrton G, et al. Clinical-grade, large-scale, feeder-free expansion of highly active human natural killer cells for adoptive immunotherapy using an automated bioreactor. Cytotherapy. 2010; 12(8):1044-55.

5. Backstrom E, Chambers B J, Ho E L, Naidenko O V, Mariotti R, Fremont D H, et al. Natural killer cell-mediated lysis of dorsal root ganglia neurons via RAE1/NKG2D interactions. European journal of immunology. 2003; 33(1):92-100.

6. Backstrom E, Chambers B J, Kristensson K, Ljunggren H G. Direct NK cell-mediated lysis of syngenic dorsal root ganglia neurons in vitro. Journal of immunology. 2000; 165(9):4895-900.

7. Hickey W F, Ueno K, Hiserodt J C, Schmidt R E. Exogenously-induced, natural killer cell-mediated neuronal killing: a novel pathogenetic mechanism. The Journal of experimental medicine. 1992; 176(3):811-7.

8. Park H, Youk J, Kim H R, Koh Y, Kwon J H, Yoon S S, et al. Infectious complications in multiple myeloma receiving autologous stem cell transplantation in the past 10 years. International journal of hematology. 2017; 106(6):801-10.

9. Park S, Jung C W, Jang J H, Kim S J, Kim W S, Kim K. Incidence of infection according to intravenous immunoglobulin use in autologous hematopoietic stem cell transplant recipients with multiple myeloma. Transpl Infect Dis. 2015; 17(5):679-87.

### EXAMPLE 2: PHARMACEUTICAL FORMULATIONS

The following examples illustrate pharmaceutical formulations according to the invention in which the active ingredient is an anti-viral agent.

### EXAMPLE A: TABLET

| | |
|---|---|
| Active ingredient | 100 mg |
| Lactose | 200 mg |
| Starch | 50 mg |
| Polyvinylpyrrolidone | 5 mg |
| Magnesium stearate | 4 mg |
| | 359 mg |

Tablets are prepared from the foregoing ingredients by wet granulation followed by compression.

### EXAMPLE B: OPHTHALMIC SOLUTION

| | |
|---|---|
| Active ingredient | 0.5 g |
| Sodium chloride, analytical grade | 0.9 g |
| Thiomersal | 0.001 g |
| Purified water to | 100 ml |
| pH adjusted to 7.5 | |

### EXAMPLE C: TABLET FORMULATIONS

The following formulations A and B are prepared by wet granulation of the ingredients with a solution of povidone, followed by addition of magnesium stearate and compression.

Formulation A

| | mg/tablet | mg/tablet |
|---|---|---|
| (a) Active ingredient | 250 | 250 |
| (b) Lactose B.P. | 210 | 26 |
| (c) Povidone B.P. | 15 | 9 |
| (d) Sodium Starch Glycolate | 20 | 12 |
| (e) Magnesium Stearate | 5 | 3 |
| | 500 | 300 |

Formulation B

| | mg/tablet | mg/tablet |
|---|---|---|
| (a) Active ingredient | 250 | 250 |
| (b) Lactose | 150 | — |
| (c) Avicel PH 101 ® | 60 | 26 |
| (d) Povidone B.P. | 15 | 9 |
| (e) Sodium Starch Glycolate | 20 | 12 |
| (f) Magnesium Stearate | 5 | 3 |
| | 500 | 300 |

Formulation C

| | mg/tablet |
|---|---|
| Active ingredient | 100 |
| Lactose | 200 |
| Starch | 50 |
| Povidone | 5 |
| Magnesium stearate | 4 |
| | 359 |

The following formulations, D and E, are prepared by direct compression of the admixed ingredients. The lactose used in formulation E is of the direction compression type.

Formulation D

| | mg/capsule |
|---|---|
| Active Ingredient | 250 |
| Pregelatinised Starch NF15 | 150 |
| | 400 |

Formulation E

| | mg/capsule |
|---|---|
| Active Ingredient | 250 |
| Lactose | 150 |
| Avicel ® | 100 |
| | 500 |

Formulation F (Controlled Release Formulation)

The formulation is prepared by wet granulation of the ingredients (below) with a solution of povidone followed by the addition of magnesium stearate and compression.

| | mg/tablet |
|---|---|
| (a) Active Ingredient | 500 |
| (b) Hydroxypropylmethylcellulose | 112 |
| (Methocel K4M Premium) ® | |
| (c) Lactose B.P. | 53 |
| (d) Povidone B.P.C. | 28 |
| (e) Magnesium Stearate | 7 |
| | 700 |

Drug release takes place over a period of about 6-8 hours and was complete after 12 hours.

### EXAMPLE D: CAPSULE FORMULATIONS

Formulation A

A capsule formulation is prepared by admixing the ingredients of Formulation D in Example C above and filling into a two-part hard gelatin capsule. Formulation B (infra) is prepared in a similar manner.

Formulation B

| | mg/capsule |
|---|---|
| (a) Active ingredient | 250 |
| (b) Lactose B.P. | 143 |
| (c) Sodium Starch Glycolate | 25 |
| (d) Magnesium Stearate | 2 |
| | 420 |

Formulation C

| | mg/capsule |
|---|---|
| (a) Active ingredient | 250 |
| (b) Macrogol 4000 BP | 350 |
| | 600 |

Capsules are prepared by melting the Macrogol 4000 BP, dispersing the active ingredient in the melt and filling the melt into a two-part hard gelatin capsule.

Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

Formulation E (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | mg/capsule |
|---|---|
| (a) Active ingredient | 250 |
| (b) Microcrystalline Cellulose | 125 |
| (c) Lactose BP | 125 |
| (d) Ethyl Cellulose | 13 |
| | 513 |

### EXAMPLE E: INJECTABLE FORMULATION

| | |
|---|---|
| Active ingredient | 0.200 g |
| Sterile, pyrogen free phosphate | 10 ml |
| buffer (pH 7.0) to | |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### EXAMPLE F: INTRAMUSCULAR INJECTION

| | |
|---|---|
| Active ingredient | 0.20 g |
| Benzyl Alcohol | 0.10 g |
| Glucofurol 75 ® | 1.45 g |
| Water for Injection q.s. to | 3.00 ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### EXAMPLE G: SYRUP SUSPENSION

| | |
|---|---|
| Active ingredient | 0.2500 g |
| Sorbitol Solution | 1.5000 g |
| Glycerol | 2.0000 g |
| Dispersible Cellulose | 0.0750 g |
| Sodium Benzoate | 0.0050 g |
| Flavour, Peach 17.42.3169 | 0.0125 ml |
| Purified Water q.s. to | 5.0000 ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further thickening is achieved as required by extra shearing of the suspension.

### EXAMPLE H: SUPPOSITORY

| | mg/suppository |
|---|---|
| Active ingredient (63 μm)* | 250 |
| Hard Fat, BP (Witepsol H15-Dynamit | 1770 |
| Nobel) | |
| | 2020 |

*The active ingredient is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 pm stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### EXAMPLE I: PESSARIES

| | mg/pessary |
|---|---|
| Active ingredient | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

1. An anti-viral agent for use in preventing a herpes virus reactivation in a patient, wherein the patient has received a therapy comprising natural killer (NK) cells and/or NK-like T cells, wherein the use comprises the step of administering the anti-viral agent to the patient with the NK cell and/or NK-like T cell therapy.

2. Use of an anti-viral agent in the manufacture of a medicament for preventing a herpes virus reactivation in a patient, wherein the patient has received a therapy comprising natural killer (NK) cells and/or NK-like T cells, wherein the anti-viral agent is administered to the patient with the NK cell and/or NK-like T cell therapy.

3. A method for preventing a herpes virus reactivation in a patient, wherein the patient has received a therapy comprising natural killer (NK) cells and/or NK-like T cells, wherein the method comprises the step of administering the anti-viral agent to the patient with the NK cell and/or NK-like T cell therapy.

4. The anti-viral agent for use of claim 1, the use of claim 2, or the method of claim 3, wherein the NK cell and/or NK-like T cell therapy induces and/or increases herpes virus reactivation.

5. The anti-viral agent for use, method, or use, of any of the preceding claims wherein the patient has a malignant disease.

6. The anti-viral agent for use, method, or use, of any of the preceding claims wherein the NK cell and/or NK-like T cell therapy is for use in the treatment of a malignant disease.

7. The anti-viral agent for use, method, or use, of claim 5 or 6 wherein the malignant disease is a haematological cancer.

8. The anti-viral agent for use, method, or use, of claim 7, wherein the haematological cancer is one selected from the group consisting of: myeloma, lymphoma, leukaemia and/or chronic myeloproliferative diseases.

9. The anti-viral agent for use, method, or use, of any of the preceding claims wherein the NK cells have the phenotype CD3⁻CD56⁺ and/or the NK-like T cells have the phenotype CD3⁺CD56⁺.

10. The anti-viral agent for use, method, or use, of any of the preceding claims wherein the NK cell and/or NK-like T cells have been expanded ex vivo.

11. The anti-viral agent for use, method, or use, of any of the preceding claims wherein the anti-viral agent is administered before and/or concurrently and/or after the patient has received the NK cell and/or NK-like T cell therapy.

12. The anti-viral agent for use, method, or use, of any of the preceding claims wherein the patient is administered the anti-viral agent at least one day before the patient receives the NK cell and/or NK-like T cell therapy, such as: at least two days before; or at least three days before; or at least four days before; or at least five days before; or at least six days before; or at least seven days before; or at least eight days before; or at least nine days before; or at least ten days before; or at least 20 days before; or at least 30 days before; or at least one month before the patient receives the NK cell and/or NK-like T cell therapy.

13. The anti-viral agent for use, method, or use, of any of the preceding claims wherein the anti-viral agent is administered at least one day after the patient received the NK cell and/or NK-like T cell therapy, such as: at least two days after; or at least three days after; or at least four days after the patient received the NK cell and/or NK-like T cell therapy.

14. The anti-viral agent for use, method, or use, of any of the preceding claims wherein 500-1500 mg of the anti-viral agent is administered to the patient within a 24 hour period.

15. The anti-viral agent for use, method, or use, of any of the preceding claims wherein the anti-viral agent is administered to the patient in one or more dose.

16. The anti-viral agent for use, method, or use, of any of the preceding claims wherein the anti-viral agent is administered to the patient in two doses of 250-750 mg in a 24 hour period, preferably wherein the anti-viral agent is administered to the patient in two doses of 500 mg in a 24 hour period.

17. The anti-viral agent for use, method, or use, of any of the preceding claims wherein the anti-viral agent is administered to the patient for a duration of at least one month, such as: at least two months; or at least three months; or at least four months; or at least five months; or at least six months; or at least seven months.

18. The anti-viral agent for use, method, or use, of any of the preceding claims wherein the patient receives NK cell and/or NK-like T cell therapy following high dose therapy (HDT) and/or autologous stem cell transplantation (ASCT).

19. The anti-viral agent for use, method, or use, of claim 18 wherein the patient receives NK cell and/or NK-like T cell therapy between three and seven months after ASCT, such as three months after ASCT, or four months after ASCT, or five months after ASCT, or six months after ASCT, or seven months after ASCT.

20. The anti-viral agent for use, method, or use, of any of the preceding claims wherein the NK cell and/or NK-like T cell therapy comprises one, or two, or three, or four, or five or more administrations of NK cells and/or NK-like T cells.

21. The anti-viral agent for use, method, or use, of any of the preceding claims wherein the NK cells and/or NK-like T cells are administered at a dosage of at least 5×10⁶ cells/kg body weight of the patient; or at least 50×10⁶ cells/kg body weight of the patient; or at least 100×10⁶ cells/kg body weight of the patient.

22. The anti-viral agent for use, method, or use, of any of the preceding claims wherein the anti-viral agent comprises a nucleoside analogue, such as one selected from the group consisting of: valacyclovir; acyclovir; famciclovir; and/or penciclovir or an active metabolite, prodrug, salt, solvate or hydrate of such nucleoside analogues.

23. The anti-viral agent for use, method, or use, of any of the preceding claims wherein the anti-viral agent is administered orally, intravenously, subcutaneously and/or intramuscularly.

24. The anti-viral agent for use, method, or use, of any of the preceding claims wherein a herpes virus selected from the group comprising: varicella zoster virus (VZV); herpes simplex virus (HSV); Epstein Barr Virus (EBV); and/or cytomegalovirus (CMV), is present in the patient.

25. The anti-viral agent for use, method, or use, of any of the preceding claims wherein the herpes virus reactivation causes shingles in the patient.

26. The anti-viral agent for use, method, or use, of any of the preceding claims wherein the patient is not lymphodepleted.

27. Natural killer (NK) cells and/or NK-like T cells for use in treating a malignant disease in a patient, wherein the use comprises the step of administering an anti-viral agent to the patient with the NK cell and/or NK-like T cell therapy.

28. Use of natural killer (NK) cells and/or NK-like T cells in the manufacture of a medicament for treating a malignant disease in a patient, wherein the patient is administered an anti-viral agent with the NK cell and/or NK-like T cell therapy.

29. A method for treating a malignant disease in a patient, comprising the step of administering a therapy comprising natural killer (NK) cells and/or NK-like T cells, and further comprising the step of administering an anti-viral agent to the patient with the NK cell and/or NK-like T cell therapy.

30. The natural killer (NK) cells and/or NK-like T cells for use, the use, or the method of any of claims 27-29, wherein the malignant disease is a haematological cancer, such as one selected from the group consisting of: myeloma, lymphoma, leukaemia and/or chronic myeloproliferative diseases.

31. The natural killer (NK) cells and/or NK-like T cells for use, the use, or the method of any of claims 27-30, wherein the anti-viral agent prevents a herpes virus reactivation in a patient.

32. The natural killer (NK) cells and/or NK-like T cells for use, the use, or the method, of any of claims 27-31 wherein the NK cell and/or NK-like T cell therapy induces and/or increases herpes virus reactivation.

33. The natural killer (NK) cells and/or NK-like T cells for use, the use, or the method of any of claims 31-32, wherein the herpes virus reactivation causes shingles in the patient.

34. The natural killer (NK) cells and/or NK-like T cells for use, the use, or the method of any of claims 27-32, wherein the patient receives NK cell and/or NK-like T cell therapy following high dose therapy (HDT) and/or autologous stem cell transplantation (ASCT).

35. The natural killer (NK) cells and/or NK-like T cells for use, method, or use of any of any of claims 27-34, wherein the anti-viral agent is as defined in any of the preceding claims.

36. The natural killer (NK) cells and/or NK-like T cells for use, method, or use of any of claims 27-35, wherein the NK cell and/or NK-like T cell therapy is as defined in any of the preceding claims.

37. A pharmaceutical composition comprising: Natural Killer (NK) cells and/or NK-like T cells as defined in any of the preceding claims, and an anti-viral agent as defined in any of the preceding claims.

38. A kit of parts comprising:
(i) a composition comprising Natural Killer (NK) cells and/or NK-like T cells, wherein the NK cells and/or NK-like T cells are as defined in any of the preceding claims; and
(ii) an anti-viral agent as defined in any of the preceding claims.

39. The pharmaceutical composition of claim 37, or the kit of claim 38 further comprising a pharmaceutically acceptable diluent, carrier or excipient.

40. An anti-viral agent for use, use, method, pharmaceutical composition, or a kit of parts, substantially as described herein with reference to the accompanying claims and examples.
